(19) United States
(12) Patent Application Publication
(10) Pub. No.: US 2018/0100199 A1
(43) Pub. Date: Apr. 12, 2018

(54) METHOD FOR DETERMINING PROGNOSIS OF BREAST CANCER PATIENT BY USING GENE EXPRESSION DATA

(71) Applicant: GWANGJU INSTITUTE OF SCIENCE AND TECHNOLOGY, Buk-gu (KR)

(72) Inventors: Hojung NAM, Buk-gu (KR); Soobok JOE, Buk-gu (KR)

(73) Assignee: GWANGJU INSTITUTE OF SCIENCE AND TECHNOLOGY, Buk-gu (KR)

(21) Appl. No.: 15/726,999

(22) Filed: Oct. 6, 2017

(30) Foreign Application Priority Data
Oct. 6, 2016 (KR) 10-2016-0129378

(51) Int. Cl. C12Q 1/68; G01N 33/574

(52) U.S. Cl. CPC C12Q 1/6886; G01N 33/57415; C12Q 2600/158; G01N 2800/7028; C12Q 2600/112

(57) ABSTRACT

A method for determining prognosis of a breast cancer patient according to an embodiment includes: collecting information on cancer tissue in a patient group from patient information determined to have breast cancer, collecting gene expression information of each patient group, sorting genes by performing a test for a group having a high expression level and a group having a low expression level based on gene expression of each patient group, clustering genes based on expression patterns of the sorted genes, defining a prognostic score using a gene expression ratio for a gene, and deriving prognosis for breast cancer in a patient group by using the prognostic score.

### CROSS-REFERENCE TO RELATED APPLICATIONS

This application claims priority to and the benefit of Korean Patent Application No. 2016-0129378, filed on Oct. 6, 2016, the disclosure of which is incorporated herein by reference in its entirety.

### BACKGROUND

### 1. Field of the Disclosure

The present disclosure relates to a method for determining prognosis of a breast cancer patient, more particularly, to a method for determining prognosis for obtaining a prognostic score by using a gene expressed in a breast cancer patient and using the score as a marker.

### 2. Discussion of Related Art

Breast cancer is ranked first in female cancer incidence and death worldwide and is the second most common tumor following thyroid cancer in female in Korea. In general, breast cancer is not a single consistent tumor, but is categorized in various aspects. In particular, a triple-negative breast cancer patient may be categorized by using a clinic test, in which estrogen receptor, progesterone receptor, and human epidermal growth factor 2 receptor do not express in tumor cells, and accounts for approximately 15 to 20% of breast cancer patients. Since there is no target responding to a particular drug, the triple-negative breast cancer is being treated with a conventional chemotherapy. In addition, a triple-negative breast cancer patient has a high rate of early recurrence and a low survival rate at an early onset of the disease. Therefore, several studies have been carried out to find various pathological characteristics found in breast cancer such as triple-negative breast cancer, and experimental models are needed to draw more accurate maps at the Omics level and to reproduce realistic aspects. In the related art, studies for prognostic analysis and prediction have identified the nature of a specific gene so as to define the gene as a prognostic gene, or the prognostic gene has been found by the comparison between the patient groups with and without a specific phenotype. For example, there is a need to find a significant prognostic gene by using statistical verification of all genes based on survival records, beyond the comparison between specific groups such as between the groups with and without metastasis and between the groups of triple-negative patients and non-triple negative patients in breast cancer.

### SUMMARY OF THE DISCLOSURE

The present disclosure is directed to providing a method for determining prognosis of a breast cancer patient capable of sorting the genes which affect prognosis of breast cancer and analyzing the prognosis of the patient, based on a gene expression level to determine prognosis of breast cancer patients using the gene expression level.

According to an aspect of the present disclosure, there is provided a method for determining prognosis of a breast cancer patient including: collecting information on cancer tissue in a patient group from patient information determined to be breast cancer; collecting gene expression information of each patient group; sorting genes by performing a test for a group having a high expression level and a group having a low expression level based on gene expression of each patient group; clustering genes based on expression patterns with respect to the sorted genes; defining a prognostic score using a gene expression ratio for a gene; and deriving prognosis for breast cancer in a patient group by using the prognostic score.

The sorting of genes by performing a test for a group having a high expression level and a group having a low expression level based on gene expression of each patient group includes performing a log-rank test, obtaining statistical significance (p-value) and hazard ratio, which indicates the survival relationship between the two groups, and determining the extent to which the expression of the gene affects survival,

Each gene is divided into two groups depending on the expression level and is composed of a group with a low survival rate when the expression level is relatively high and a group with a low survival rate when the expression level is relatively low.

The clustering of genes based on expression patterns of the sorted genes includes creating a maximal clique for the genes corresponding to two groups and clustering gene graphs by using Pearson correlation coefficient.

The Pearson correlation coefficient (similarity) between the genes corresponding to the cluster of a group with a poor survival rate as the expression level is high and the cluster of a group with a poor survival rate as the expression level is low is calculated, and a gene with opposite expression is found.

The prognostic score is set to a value obtained by dividing an average value of a gene expression profile having a high expression level in a patient having a low survival rate by an average value of a gene expression profile having a low expression level in a patient having a low survival rate.

The deriving of prognosis for breast cancer in a patient group by using the prognostic score includes determining that the prognosis for breast cancer is not good when the prognostic score is relatively high.

According to an aspect of the present disclosure, genetic markers for prediction of prognosis of breast cancer may be identified and used to design a prognostic score, and thus information on therapeutic drug targets and personalized treatment can be presented in future.

### BRIEF DESCRIPTION OF THE DRAWINGS

FIG. 1 is a flowchart of a method for determining prognosis of a breast cancer patient by using gene expression data according to an embodiment.

FIG. 2 is a schematic view illustrating a process of sorting genes affecting a survival rate of a patient.

FIG. 3 is a graph illustrating a survival rate of a patient against time by a log-rank test.

FIG. 4 is a view illustrating gene clustering by using a maximal clique.

FIG. 5 is a graph illustrating a survival rate of a patient based on a prognostic score.

FIG. 6 is a graph illustrating a distribution of a prognostic score depending on the nature of a breast cancer patient.

### DETAILED DESCRIPTION OF EXEMPLARY EMBODIMENTS

Exemplary embodiments of the present disclosure will be described in detail below with reference to the accompanying drawings. While the present disclosure is shown and described in connection with exemplary embodiments thereof, it will be apparent to those skilled in the art that various modifications can be made without departing from the spirit and scope of the disclosure.

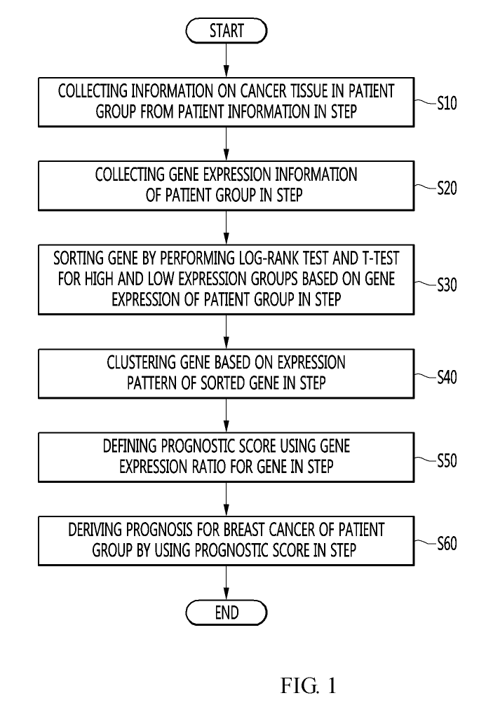

FIG. 1 illustrates a flowchart of a method for determining prognosis of a breast cancer patient by using gene expression data according to an embodiment.

Referring to FIG. 1, a method for determining prognosis of a breast cancer patient according to an embodiment may be performed including: collecting information on a cancer tissue of a patient group from patient information which is known to have cancer in step S10, collecting gene expression information of a patient group in step S20, sorting genes by performing a log-rank test and a t-test for high and low expression groups based on the gene expression of the patient group in step S30, clustering the genes based on the expression pattern of the sorted genes in step S40, defining a prognostic score using a gene expression ratio for the genes in step S50, and deriving prognosis for the breast cancer of the patient group by using the prognostic score in step S60. Next, characteristics will be described in each step.

A gene used in an embodiment may be any genes that may be measured by a micro-array or RNA-seq. In an embodiment, first, the step S10 of collecting information on cancer tissues of a patient group from patient information which is known to have cancer and the step S20 of collecting gene expression information of a patient group are performed.

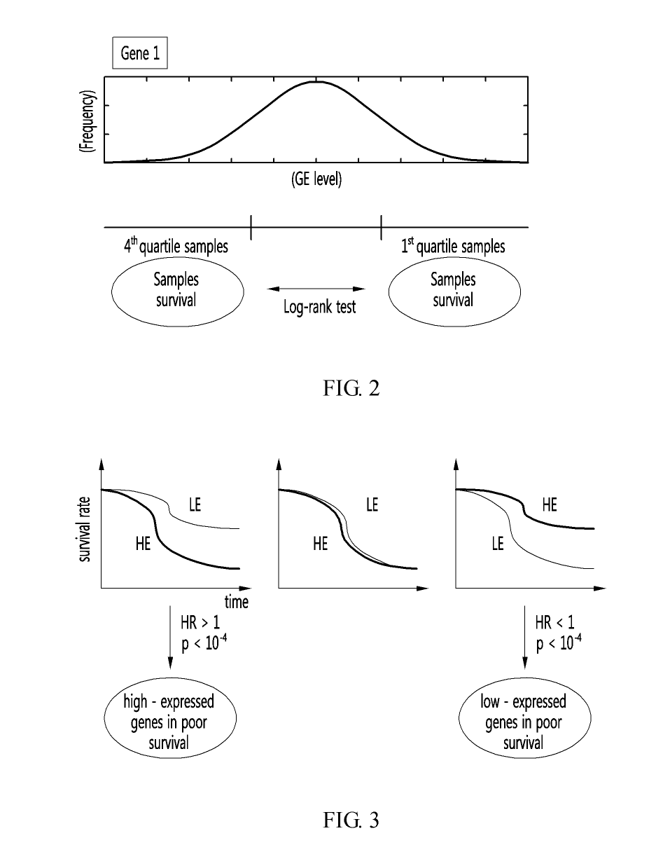

FIG. 2 is a schematic view illustrating a process of sorting genes affecting a survival rate of a patient. Referring to FIG. 2, frequency against a gene expression level (GE level) of Gene 1 is illustrated.

The step S30 of sorting genes by performing the log-rank test and the t-test for high and low expression groups based on the gene expression of the patient group is as follows.

First, patients are listed in descending order of expression level for each gene, then the log-rank test and the t-test are performed on the high and low patient groups. The log-rank test is a hypothetical verification method for comparing the survival distributions of two samples and is a nonparametric verification method suitable for the cases where data is distorted or measuring values are only partially known. In an embodiment, 25% high and 25% low patient groups are defined, and the level may be appropriately changed depending on user's definitions.

FIG. 3 is a graph illustrating a survival rate of a patient against time by a log-rank test. Referring to FIG. 3, graphs are Kaplan-Meier survival curves illustrating a gene expression level on survival by using a statistical significance (p-value) derived from a log rank test and a hazard ratio representing the survival relationship between two groups.

When performing a log-rank test, the result as many as the number of genes performed may be derived, and the significance (p-value) subsequent to each log-rank test of each gene to find a gene related to survival as a prognostic factor may be calculated. In an embodiment, the p-value is cut off to be 10⁻¹ or less.

Each gene may be divided into two groups depending on the expression level, that is, high-expressed (HE) genes in poor survival when the expression level is relatively high, and low-expressed (LE) genes in poor survival when the expression level is relatively low.

Next, the step S40 of clustering genes based on expression patterns of the sorted genes is as follows. Since the genes extracted by using the method described above differ only in the expression level of each gene, when the clustering method is applied to the entire sample, the genes cluster depending on the extent of similarity of the expression level.

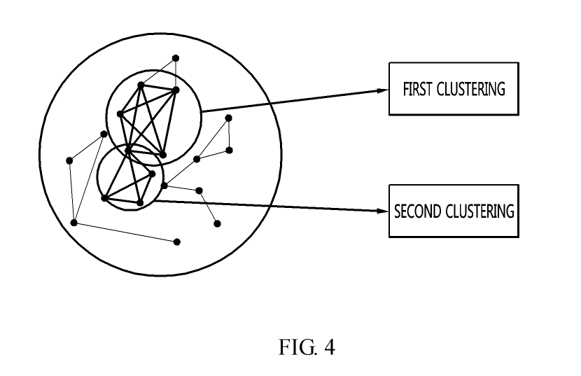

FIG. 4 is a view illustrating gene clustering by using a maximal clique.

Referring to FIG. 4, a gene graph is constructed for genes whose survival rate is low when expression is high or low. Each vertex on the graph represents a gene, an edge is created depending on the extent of similarity of the gene expression, and a mathematical value is used to determine the similarity between two vectors. Various correlation coefficients may be used. In an embodiment, the extent of similarity is determined by using Pearson correlation coefficient, and the Pearson correlation coefficient may be represented in the following equation.

$${r}_{X , Y} = \frac{E [ ( X - {μ}_{X} ) ( Y - {μ}_{Y} ) ]}{{σ}_{X} {σ}_{Y}} , \\ E [ ( X - {μ}_{X} ) ( Y - {μ}_{Y} ) ] = \frac{{\sum }_{i = 1}^{m} ( {x}_{i} - {μ}_{X} ) ( {y}_{i} - {μ}_{Y} )}{m} \qquad [ Equation 1 ]$$

Here, r_{X,Y} represents Pearson correlation coefficient of the genes X and Y, respectively, where μ_{X} is an average of the X gene expression profile, μ_{Y} is an average of the Y gene expression profile, and σ_{X} and σ_{Y} represent a standard deviation of the genes X and Y, respectively. m refers to a total number of patient samples.

In an embodiment, the gene graph constructed by using the Pearson correlation coefficient value is used for clustering. First, a largest maximal clique is found using a maximal clique algorithm, and a first cluster belongs thereto is defined.

All connections found in the graph is cleared, then a maximal clique is found again, then a second cluster belongs thereto is defined. The process is applied to both two gene groups obtained in step S30, and individual clustered gene sets may be found in both high and low gene expression groups.

If two gene groups are clustered as described in step S40, mutually inversely expressed gene sets subject to genes among clusters may be found.

If the Pearson correlation coefficient (similarity) between the genes of cluster A in which a survival rate of the group is low as the expression is high and the genes of cluster A′ in which a survival rate of the group is low as the expression is low is calculated. Since a correlation coefficient value is low as the expression pattern is opposite, in this way, genes with opposite expression may be found, which may be used as an important gene in a scale used for prognostic analysis.

In an embodiment, a maximal bi-clique algorithm is used in the process. If a graph is constructed between the genes belonging to the clusters A and A′, each of the connecting edges is determined by a Pearson correlation coefficient value, and if a maximal clique is founded, the gene set used for the final prognostic analysis may be found.

The step S50 of defining a prognostic score using a gene expression ratio for the genes is as follows.

If a final gene is sorted in step S40, the genes may be sorted as a gene with lower survival as the expression is higher or lower survival as the expression is lower. In an embodiment, a prognostic score S is defined as a ratio of the averages of two gene sets, and an average value of expression profile of highly expressed genes GEH in patients with a low survival rate may be divided by an average value of expression profile of lowly expressed genes GEL in patients with a low survival rate.

In an embodiment, the step S60 of deriving prognosis for breast cancer in a patient group may be performed depending on the prognostic score.

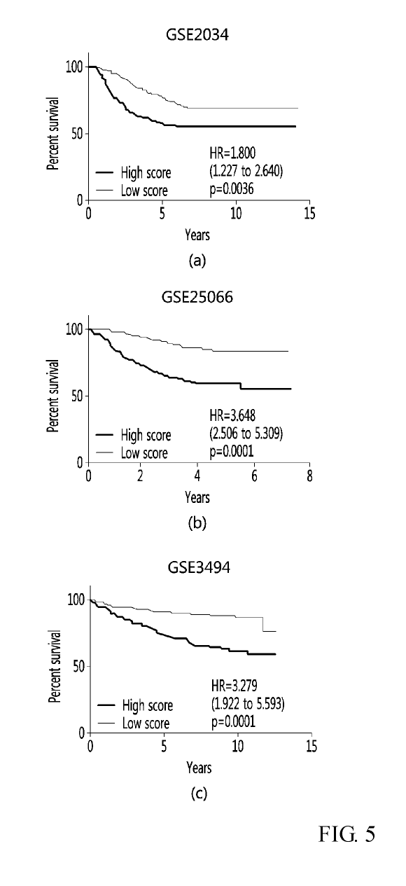

FIG. 5 is a graph illustrating a survival rate of a patient based on a prognostic score.

Referring to FIG. 5, there are graphs in which verifications are performed using three independent array data to determine whether the prognostic score defined in step S50 is applicable to prognosis of other patients. The data set used is from the GEO database, and (a), (b), and (c) is the array information of GSE25066, GSE3494, and GSE2034, respectively.

For each data, patients are divided into two groups based on the prognostic score, and Kaplan-Meier survival curves for the two patient groups are drawn.

Referring to the graphs of FIG. 5, it can be seen that the prognosis of the patients with a low score is better than that of the patients with a high score. Since a significant p-value is represented in (a) to (c), respectively, it can be seen that the prognostic score defined in an embodiment may be used to predict the prognosis of breast cancer.

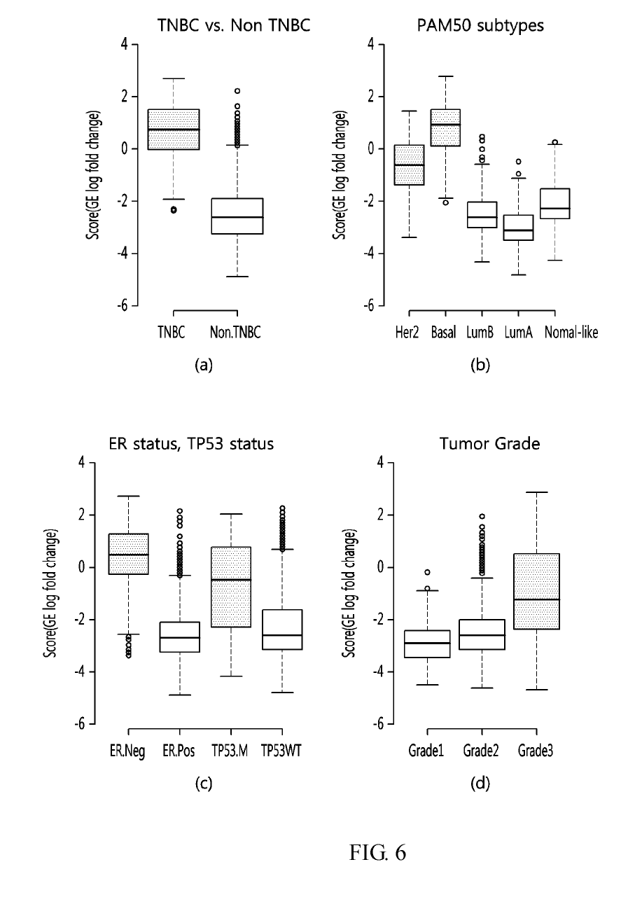

It can be seen that the score varies depending on a particular nature of breast cancer patients, which is higher in breast cancer patients with poor prognosis. FIG. 6 is a graph illustrating a distribution of a prognostic score depending on the nature of a breast cancer patient.

Breast cancer is classified into several categories according to a category criteria. Typically, breast cancer may be classified into hormone receptor-positive breast cancer, HER-2 gene-positive breast cancer, and triple-negative breast cancer (TNBC), in which both the hormone receptor and the HER-2 gene are negative. According to PAM 50 category criteria, breast cancer may be divided into HER2-enriched, Basal-like, Luminal A, and Luminal B. In addition, a tumor grade, mutation status of TP53 gene, and the like may be included in the category criteria depending on a condition of each patient.

Referring to FIG. 6, (a) is a graph illustrating a prognostic score in the case of breast cancer corresponding to triple-negative breast cancer and in the case of breast cancer corresponding to non-triple negative breast cancer. In the case of breast cancer corresponding to the triple-negative breast cancer, it can be seen that the center value of the prognostic score is distributed at 0.8, but in the other cases, it can be seen that the center value of the prognostic score is distributed at the vicinity of −2.2. Triple-negative breast cancer is known as an existing high-risk breast cancer. As the result of deriving a prognostic score in the method for determining prognosis of breast cancer patients according to an embodiment, since it can be seen that the prognostic score in the triple-negative breast cancer is higher than other cases, it can be determined that the prognostic score of the present disclosure represents a risk for the existing high-risk group of patients.

As shown in the graph of (b), it can be seen that the prognostic scores of HER2-enriched and basal-like patient groups are also significantly higher than those of other patient groups according to the PAM 50 category. As shown in (c), it can be seen that patients in the estrogen receptor negative group and TP53 gene mutation group have a high prognostic score. As shown in (d), it can be seen that a prognostic score is higher as a tumor differentiation degree of cancer is high (more malignant).

That is, it can be confirmed that prediction of prognosis of breast cancer by using a prognostic score of an embodiment has a high value for the known breast cancer, and it can be seen that an unknown gene can be determined by using the prognostic score.

In an embodiment, a genetic marker is identified depending on the gene expression level expressed by breast cancer, and used to design a prognostic score, and thus the prognostic score can be used to advantage in presenting information necessary for a future therapeutic drug target and customized treatment.

According to an aspect of the present disclosure, genetic markers for prediction of prognosis of breast cancer may be identified and used to design a prognostic score, and thus information on therapeutic drug targets and personalized treatment can be presented in future.

It will be apparent to those skilled in the art that various modifications can be made to the above-described exemplary embodiments of the present disclosure without departing from the spirit or scope of the disclosure. Thus, it is intended that the present disclosure covers all such modifications provided they come within the scope of the appended claims and their equivalents.

What is claimed is:

1. A method for determining prognosis of a breast cancer patient, comprising:
collecting information on cancer tissue in a patient group from patient information determined to have breast cancer;
collecting gene expression information of each patient group;
sorting genes by performing a test for a group having a high expression level and a group having a low expression level based on gene expression of each patient group;
clustering genes based on expression patterns of the sorted genes;
defining a prognostic score using a gene expression ratio for a gene; and
deriving prognosis for breast cancer in a patient group by using the prognostic score.

2. The method for determining prognosis of a breast cancer patient of claim 1, wherein the sorting of genes by performing a test for a group having a high expression level and a group having a low expression level based on gene expression of each patient group comprises performing a log-rank test, obtaining statistical significance (p-value) and hazard ratio, which indicates the survival relationship between the two groups, and determining the extent to which the expression of the gene affects survival.

3. The method for determining prognosis of a breast cancer patient of claim 2, wherein each gene is divided into two groups depending on the expression level and is composed of a group with a low survival rate when the expression level is relatively high and a group with a low survival rate when the expression level is relatively low.

4. The method for determining prognosis of a breast cancer patient of claim 1, wherein the clustering of genes based on expression patterns of the sorted genes comprises creating a maximal clique for the genes corresponding to two groups and clustering gene graphs by using Pearson correlation coefficient.

5. The method for determining prognosis of a breast cancer patient of claim 4, wherein the Pearson correlation coefficient (similarity) between the genes corresponding to the cluster of a group with a poor survival rate as the expression level is high and the cluster of a group with a poor survival rate as the expression level is low is calculated, and a gene with opposite expression is found.

6. The method for determining prognosis of a breast cancer patient of claim 1, wherein the prognostic score is set to a value obtained by dividing an average value of a gene expression profile having a high expression level in a patient having a low survival rate by an average value of a gene expression profile having a low expression level in a patient having a low survival rate.

7. The method for determining prognosis of a breast cancer patient of claim 6, wherein the deriving of prognosis for breast cancer in a patient group by using the prognostic score comprises determining that the prognosis for breast cancer is not good when the prognostic score is relatively high.
